(19) United States
(12) Patent Application Publication
(10) Pub. No.: US 2024/0013498 A1
(43) Pub. Date: Jan. 11, 2024

(54) INFORMATION PROCESSING DEVICE, INFORMATION PROCESSING METHOD, AND INFORMATION PROCESSING PROGRAM

(71) Applicant: SONY GROUP CORPORATION, TOKYO (JP)

(72) Inventor: YUGO SATO, TOKYO (JP)

(21) Appl. No.: 18/254,885

(22) PCT Filed: Oct. 11, 2021
(86) PCT No.: PCT/JP2021/037520

§ 371 Date: May 30, 2023

(30) Foreign Application Priority Data
Dec. 4, 2020 (JP) 2020-201507

(51) Int. Cl. G06T 19/20; G09G 3/00; G06F 3/01

(52) U.S. Cl. CPC G06T 19/20; G09G 3/001; G06F 3/011; G09G 2354/00; G06T 2219/2016; G09G 2340/04

(57) ABSTRACT

[Object]

Provided are an information processing device, an information processing method, and an information processing program that are new, improved, and able to modify the length of a part of a model in an easier manner. [Solving Means] An information processing device includes a display control section and a modification section. The display control section generates a display screen. The generated display screen contains a video image of a target part. The target part is one of the parts included in a model, is fixed in length in the video image, and corresponds to a first part of a user. The modification section modifies the length of the target part of the model in reference to a first distance at a first time point. The first distance is determined by a distance sensor and is indicative of the distance between the distance sensor and the first part of the user. The first time point is a point of time when the video image of the target part apparently matches the length of the first part of the user.

### TECHNICAL FIELD

The present disclosure relates to an information processing device, an information processing method, and an information processing program.

### BACKGROUND ART

In recent years, a technology for modifying a hand model in which the hand shape of a user is reflected has been developed with the spread of AR (Augmented Reality) and VR (Virtual Reality). A technology disclosed, for example, in PTL 1 enables a touchscreen mounted on electronic equipment to detect a contact area between the touchscreen and a finger of the user when the user presses the finger against the touchscreen, and then calculate the length of the nail of the finger in reference to the contact area and a captured image of the finger performing a touch operation.

### CITATION LIST

### Patent Literature

[PTL 1]
Japanese Patent Laid-open No. 2015-149036

### SUMMARY

### Technical Problem

However, the technology described in PTL 1 requires the use of equipment having a touchscreen and a camera. Further, the user has to press the finger against the touchscreen and capture an image of the finger by the camera.

In view of the above circumstances, the present disclosure proposes a new, improved information processing device that makes it easier to modify the length of a part of a model.

### Solution to Problem

According to an aspect of the present disclosure, there is provided an information processing device including a display control section and a modification section. The display control section generates a display screen. The generated display screen contains a video image of a target part. The length of the target part is fixed in the video image. The target part is one of the parts included in a model and corresponds to a first part of a user. The modification section modifies the length of the target part of the model in reference to a first distance at a first time point. The first distance is determined by a distance sensor and is indicative of the distance between the distance sensor and the first part of the user. The first time point is a point of time when the video image of the target part apparently matches the length of the first part of the user.

According to another aspect of the present disclosure, there is provided a computer-executed information processing method including generating a display screen that contains a video image of a target part, the length of the target part being fixed in the video image, and the target part being one of the parts included in a model and corresponding to a first part of a user, and modifying the length of the target part of the model in reference to a first distance at a first time point, the first distance being determined by a distance sensor and indicative of the distance between the distance sensor and the first part of the user, and the first time point being a point of time when the video image of the target part apparently matches the length of the first part of the user.

According to still another aspect of the present disclosure, there is provided an information processing program that causes a computer to function as a display control section and as a modification section. The display control section generates a display screen. The generated display screen contains a video image of a target part. The length of the target part is fixed in the video image. The target part is one of the parts included in a model and corresponds to a first part of a user. The modification section modifies the length of the target part of the model in reference to a first distance at a first time point. The first distance is determined by a distance sensor and is indicative of the distance between the distance sensor and the first part of the user. The first time point is a point of time when the video image of the target part apparently matches the length of the first part of the user.

### BRIEF DESCRIPTION OF DRAWINGS

FIG. 1 is an explanatory diagram illustrating an overview of an information processing system according to the present disclosure.

FIG. 2 is a block diagram illustrating a functional configuration of an information processing device 10 according to the present disclosure.

FIG. 3 is an explanatory diagram illustrating an example of a method for acquiring distance information.

FIG. 4 is an explanatory diagram illustrating an example of a method for detecting feature points from a depth image.

FIG. 5 is an explanatory diagram illustrating an example of a method for modifying the scale of a hand model by using a model customization processing section 121.

FIG. 6 is an explanatory diagram illustrating an example of a method for modifying the length of a target part of a hand model by using the model customization processing section 121.

FIG. 7 is an explanatory diagram illustrating an example of a method for modifying the length of a target part of the hand model by using the model customization processing section 121.

FIG. 8 is an explanatory diagram illustrating an example of a method for modifying the widths for fingers of the hand model by using the model customization processing section 121.

FIG. 9 is an explanatory diagram illustrating the operations of the information processing system according to the present disclosure.

FIG. 10 is an explanatory diagram illustrating an example flow for modifying the scale of a hand model by using a first scale modification method according to the present disclosure.

FIG. 11 is an explanatory diagram illustrating an example flow for modifying the scale of a hand model by using a second scale modification method according to the present disclosure.

FIG. 12 is an explanatory diagram illustrating an example flow for modifying the length of a finger of a hand model according to the present disclosure.

FIG. 13 is an explanatory diagram illustrating an example flow for modifying the width for a finger of a hand model according to the present disclosure.

FIG. 14 is a block diagram illustrating an example hardware configuration of the information processing device 10 according to the present disclosure.

### DESCRIPTION OF EMBODIMENT

A preferred embodiment of the present disclosure will now be described in detail with reference to the accompanying drawings. It should be noted that, in this document and the accompanying drawings, constituent elements having substantially the same functional configuration are designated by the same reference signs and will not be redundantly described.

It should be noted that the description will be given in the following order.
1. Overview
2. Example Configuration
2.1. Example Configuration of Information Processing Device
2.2. Details of Model Customization Processing
3. Examples of Operation Processing
4. Examples of Operational Advantages
5. Example Hardware Configuration of Information Processing Device 10 according to Present Disclosure
6. Supplement

### <1. Overview>

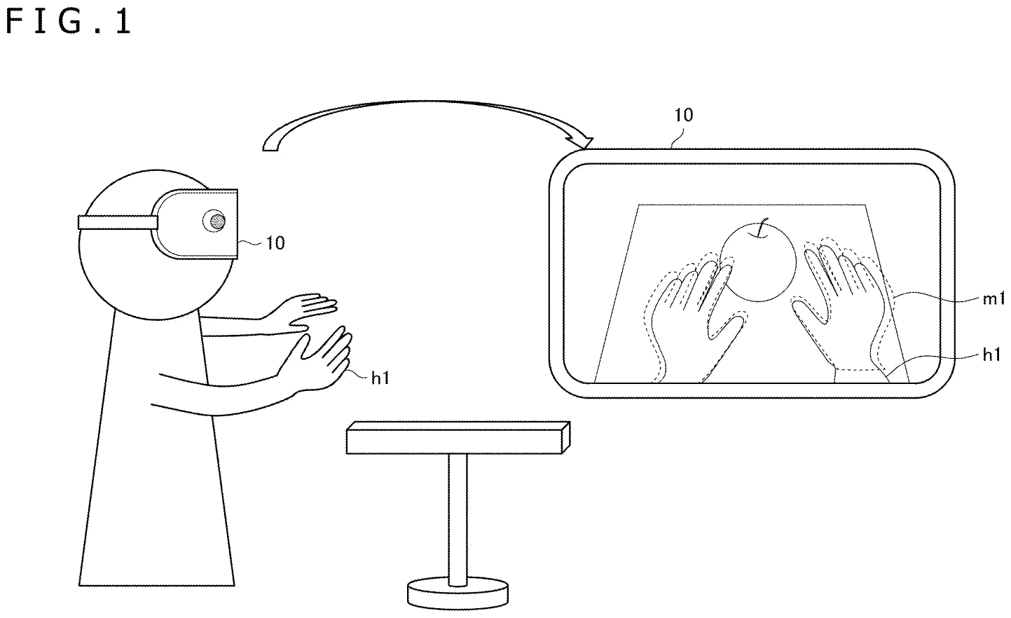

An embodiment of the present disclosure relates to an information processing system that makes it easier to modify the length of a part of a model. The information processing system will now be outlined with reference to FIG. 1.

FIG. 1 is an explanatory diagram illustrating an overview of the information processing system according to the present disclosure. As depicted in FIG. 1, the information processing system according to the present disclosure includes, for example, an information processing device 10.

The information processing device 10 may be, for example, an HMD (Head Mounted Display) or smart glass with AR technology. In this case, the information processing device 10 provides a user with various types of content through such a display.

As depicted, for example, in FIG. 1, the user wearing the information processing device 10 is able to view, through the display, a screen displaying a virtual object (e.g., an apple) placed on a table that is in reality not placed on the table.

Further, a hand model m1 is superimposed, in the background, on a hand of the user wearing the information processing device 10. The user is able, for example, to select or operate the displayed virtual object through the hand model m1 superimposed on a hand h1 of the user in the background.

It should be noted that, for example, the position and posture of the hand model m1 are determined according, for example, to the position of the hand h1 of the user or the posture of the user.

Various parameters, such as a finger length and a hand scale, are set for the hand model m1 superimposed on a hand of the user. However, such parameters may vary depending on the user's hand to be used. In a case where the hand model m1 is misaligned with the hand h1 of the user, the user may feel some kind of strangeness when selecting or operating the virtual object.

Consequently, it is desirable that various parameters related to the misalignment between the hand h1 of the user and the hand model m1 superimposed on the user's hand be modified in order to provide improved user-friendliness.

Accordingly, in an embodiment according to the present disclosure, the information processing device 10 is configured to modify the various parameters.

### (Information Processing Device 10)

When a misalignment occurs between a part of the user and a target part of the model, which is one of the parts included in the model and corresponds to the part of the user, the information processing device 10 modifies the target part of the model in reference to information acquired from the information processing device 10 or from another mobile terminal.

It should be noted that, unless otherwise stated, this document describes an optical transmission type HMD with AR technology as the information processing device 10. However, the present disclosure is not limited to any specific example.

For example, multiple different display methods, such as a non-transmissive type, a video transmission type, and an optical transmission type, are available as the display method for the HMD. However, the HMD may adopt any of such display methods.

Further, the embodiment of the present disclosure is applicable to equipment with VR technology (e.g., HMD) and mobile terminals such as smartphones and cameras.

Further, the information processing device 10 may be a server that is connected, for example, to an HMD or a mobile terminal through a network. The network may include, for example, the Internet, a leased line, a LAN (Local Area Network), or a WAN (Wide Area Network). In such a case, the information processing device 10 may receive later-described information required for model modification, for example, from the HMD. Subsequently, the information processing device 10 may modify the model in reference to the information received, for example, from the HMD, and transmit the modified model, for example, to the HMD.

A configuration and operations of the information processing device 10 according to the present disclosure will now sequentially be described in detail.

### <2. Example Configuration>

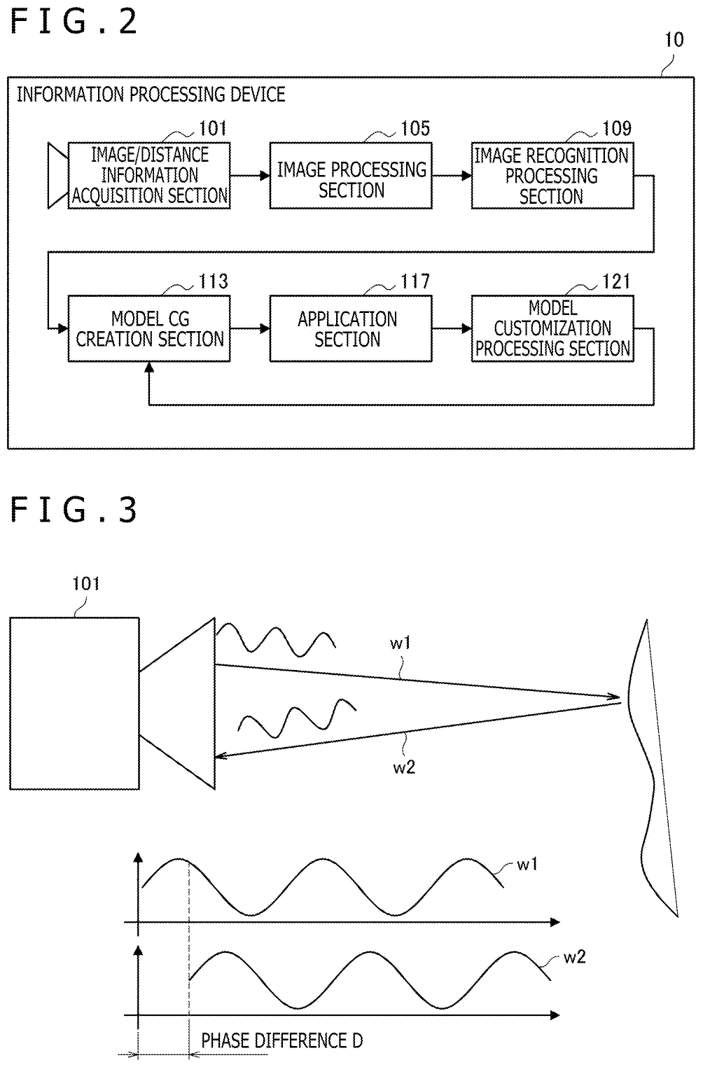

FIG. 2 is a block diagram illustrating a functional configuration of the information processing device 10 according to the present disclosure. As depicted in FIG. 2, the information processing device 10 includes an image/distance information acquisition section 101, an image processing section 105, an image recognition processing section 109, a model CG creation section 113, an application section 117, and a model customization processing section 121.

### (Image/Distance Information Acquisition Section 101)

The image/distance information acquisition section 101 has a function of capturing an image of a subject to acquire image information or distance information. The image information or the distance information may be, for example, RAW data that is a set of electrical signals obtained by image capture.

Further, the image/distance information acquisition section 101 may include, for example, a CCD sensor or a CMOS sensor to implement the function of acquiring the image information or include a distance sensor (e.g., ToF sensor) to implement the function of acquiring the distance information. An example of distance information acquisition by the image/distance information acquisition section 101 will now be described with reference to FIG. 3.

FIG. 3 is an explanatory diagram illustrating an example of a method for acquiring the distance information. With reference to FIG. 3, an example of a method for acquiring the distance information through the use of a ToF camera that adopts an indirect ToF (iToF: indirect Time Flight) method is described below as an example of the image/distance information acquisition section 101. The upper part of the graph in FIG. 3 depicts the waveform of a radiation wave w1 emitted from the image/distance information acquisition section 101, whereas the lower part of the graph depicts the waveform of a reflected wave w2 that is generated when the radiation wave w1 is reflected by a target.

The image/distance information acquisition section 101 may include, for example, a light-emitting section and a light-receiving section. In this case, the light-emitting section may emit the radiation wave w1 toward the target, and the light-receiving section may receive the reflected wave w2 that is generated when the radiation wave w1 is reflected by the target. In this instance, the phase of the reflected wave w2 received by the light-receiving section varies with the distance from the position of the distance sensor to the target.

Consequently, the image/distance information acquisition section 101 is able to detect the distance between the distance sensor position and the target in reference to a phase difference D between the emitted radiation wave w1 and the received reflected wave w2.

It should be noted that an example of a method for acquiring distance information through the use of the indirect ToF method has been described with reference to FIG. 3. However, the method for acquiring the distance information is not limited to the method described in the above example. The distance information may be acquired with use of an alternative method such as a direct ToF (dToF: direct Time of Flight) method, a stereotype method, or a structured-light method.

### (Image Processing Section 105)

The image processing section 105 performs a process of converting an electrical signal containing the image information acquired by the image/distance information acquisition section 101 to a digital image and converting an electrical signal containing the distance information to a depth image. The image processing section 105 performs, for example, ISP (Image Signal Processing) on the acquired RAW data in order to convert the RAW data to various images.

### (Image Recognition Processing Section 109)

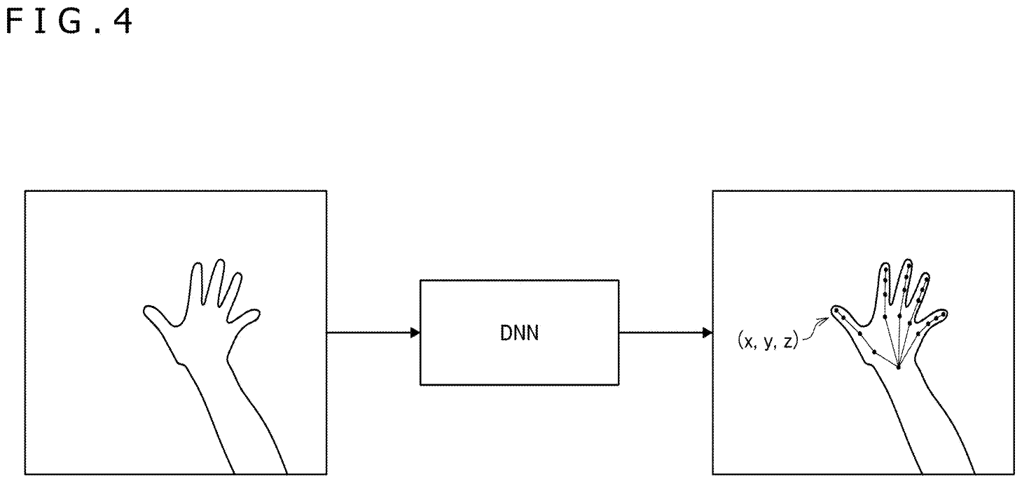

The image recognition processing section 109, which is an example of a detection section, performs a process of detecting feature points from image data obtained by the image processing section 105. An example of the process of detecting the feature points is described below with reference to FIG. 4.

FIG. 4 is an explanatory diagram illustrating an example of a method for detecting the feature points from the depth image. The image/distance information acquisition section 101 captures an image, for example, of a hand of the user to acquire an electrical signal containing the distance information. Next, the image processing section 105 performs the process of converting the electrical signal containing the distance information acquired by image capture to the depth image. Subsequently, the image recognition processing section 109 may detect three-dimensional position coordinates of the feature points (e.g., hand joint points) from the depth image by using, for example, a machine learning technology such as a DNN (Deep Neural Network).

Further, the image recognition processing section 109, which is an example of a virtual line generation section, may generate a virtual line by connecting recognized feature points. For example, the image recognition processing section 109 may generate the virtual line by connecting the joint points of each recognized finger.

It should be noted that the example of the method for detecting the feature points by applying the DNN or other machine learning technology to the depth image has been described with reference to FIG. 4. However, the three-dimensional position coordinates of the feature points may be detected by applying the DNN or other machine learning technology to a digital image.

### (Model CG Creation Section 113)

The model CG creation section 113 estimates model parameters in reference to the feature points detected by the image recognition processing section 109, and creates a model corresponding to the parts of the user in reference to the estimated model parameters.

For example, the model CG creation section 113 estimates the scale value of a hand as a model parameter in accordance with the hand's feature points detected by the image recognition processing section 109. Subsequently, the model CG creation section 113 creates a hand model corresponding to the user's hand in reference to the estimated scale value of the hand.

### (Application Section 117)

The application section 117 generates a display screen that contains the model created by the model CG creation section 113. For example, the application section 117 generates the display screen in which the user's hand model created by the model CG creation section 113 is superimposed on the user's hand that is visible through a lens.

It should be noted that the application section 117 may or may not cause the display screen to display the model created by the model CG creation section 113. In a case where the model is not to be displayed on the display screen, the application section 117 may use, for example, the created model in the background.

### (Model Customization Processing Section 121)

When a misalignment occurs between a part of the user and a model corresponding to that part, the model customization processing section 121 performs a process of modifying the model. For example, in a case where a misalignment occurs when the user's hand model created by the model CG creation section 113 is superimposed on the user's hand, the model customization processing section 121 performs the process of modifying the model.

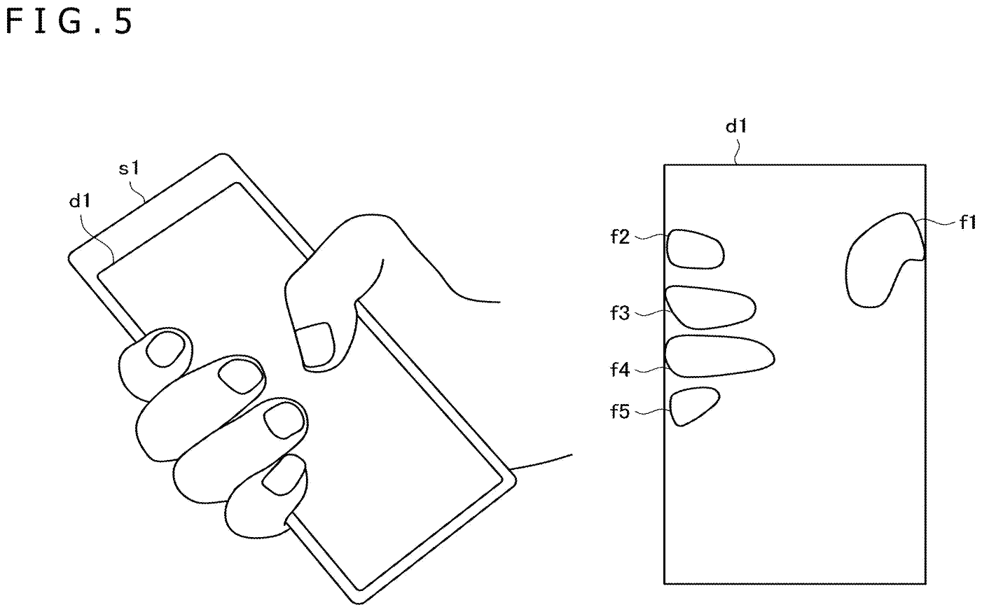

More specifically, in a case where a misalignment occurs between the user's hand and the hand model, the model customization processing section 121 performs the process of modifying the hand model by modifying various parameters related to the misalignment. For example, the model customization processing section 121 may perform the process of modifying at least one of the scale of the hand model, the finger length of the hand model, and the finger width of the hand model. A method for modifying the various parameters is described in detail below with reference to FIGS. 5 to 8.

### (Scale Modification of Hand Model)

The model customization processing section 121, which functions as a contact area calculation section, calculates the contact area of a specific part when the mobile terminal is grasped by the user. Further, the model customization processing section 121 may modify the scale of the hand model in reference to the calculated contact area.

Moreover, the model customization processing section 121, which is an example of the combination of a storage section and a magnification calculation section, may pre-store an average contact area of the hand model having a certain scale, and calculate a scale magnification in reference to the average contact area and the calculated contact area. Further, the model customization processing section 121 may modify the scale of the hand model in reference to the pre-stored scale of the hand model and the calculated scale magnification. An example of a method adopted by the model customization processing section 121 to modify the scale of the hand model is specifically described below with reference to FIG. 5.

FIG. 5 is an explanatory diagram illustrating an example of the method for modifying the scale of the hand model by using the model customization processing section 121. The left part of FIG. 5 depicts a smartphone s1 grasped by the user, whereas the right part of FIG. 5 depicts the contact surface between finger pads of the user and a touch display d1. It should be noted that the finger pads are simply referred to as the fingers in the following description.

First, as depicted in FIG. 5, the user grasps the smartphone s1 including the touch display d1. Then, the smartphone s1 transmits, to the information processing device 10, contact information regarding the contact between the touch display d1 and the fingers of the user.

Next, the model customization processing section 121 calculates the contact area between the user's fingers and the touch display d1 from the contact information received from the smartphone s1. More specifically, the model customization processing section 121 calculates the contact area between the user's fingers and the touch display d1 by adding up the contact areas of all of the user's fingers placed in contact with the touch display d1, namely, a contact area f1 of a thumb, a contact area f2 of an index finger, a contact area f3 of a middle finger, a contact area f4 of a ring finger, and a contact area f5 of a little finger. The contact area between the user's fingers and the touch display d1 is hereinafter simply referred to as the contact area.

As a first scale modification method, the model customization processing section 121 may modify the hand model into a hand model having a first scale in a case where the contact area is equal to or greater than a threshold, and may modify the hand model into a hand model having a second scale in a case where the contact area is smaller than the threshold. The second scale is smaller than the first scale.

It should be noted that the model customization processing section 121 may perform modification such that the hand model having the first scale is regarded as a hand model representing an adult's hand and that the hand model having the second scale is regarded as a hand model representing a child's hand. For example, in the case where the contact area is equal to or greater than the threshold, the model customization processing section 121 modifies various hand model parameters into the scale, finger lengths, and finger widths prepared as adult hand model parameters. Further, in the case where the contact area is smaller than the threshold, the model customization processing section 121 modifies the various hand model parameters into the scale, finger lengths, and finger widths prepared as child hand model parameters.

Further, the information processing device 10 may change a recognition mode as needed depending on whether the hand model is modified into the first scale hand model or into a second scale hand model. For example, in a case where the hand model is modified into the first scale hand model, the information processing device 10 may change the recognition mode into a crop scale corresponding to the first scale. Meanwhile, in a case where the hand model is modified into the second scale hand model, the information processing device 10 may change the recognition mode into a crop scale corresponding to the second scale. This enables the information processing device 10 to improve, for example, the accuracy of recognition of the user's hand.

Moreover, the model customization processing section 121 may prepare three or more hand models that differ in scale. In such a case, each of the prepared hand models has a contact area determination range corresponding to the scale of each of the prepared hand models. The model customization processing section 121 modifies the hand model into a hand model that is determined as being scaled within the contact area determination range.

As a second scale modification method, the model customization processing section 121 may associate, for example, the hand model having the first scale with the average contact area in the case where the smartphone s1 is grasped by a hand having the first scale, and pre-store the resulting association.

Further, the model customization processing section 121 may calculate, as the scale magnification, the ratio between the pre-stored average contact area and the contact area calculated when the smartphone s1 is grasped by the user. Subsequently, the model customization processing section 121 may modify the scale of the hand model by multiplying a first scale value by the calculated scale magnification.

The method for modifying the scale of the hand model has been specifically described above. However, the contact area to be used for scale modification of the hand model may be the contact area obtained when the smartphone s1 is grasped intentionally by the user for hand model scale modification or the contact area obtained when the smartphone is grasped by the user in everyday life.

### (Length Modification of Target Part of Hand Model)

An example of a method adopted by the model customization processing section 121 to modify the length of a target part of the hand model will now be described.

The model customization processing section 121, which functions as a display control section and as a modification section, generates a display screen that contains a video image of a target part corresponding to a desired part of the user, which is one of the user's fingers included in the hand model, while the length of the target part is fixed in the video image.

Further, the model customization processing section 121 modifies the length of the target part of the model in reference to a first distance. The first distance is the distance between the distance sensor and the desired part of the user and determined at a point of time when the video image of the target part apparently matches the length of the desired part of the user.

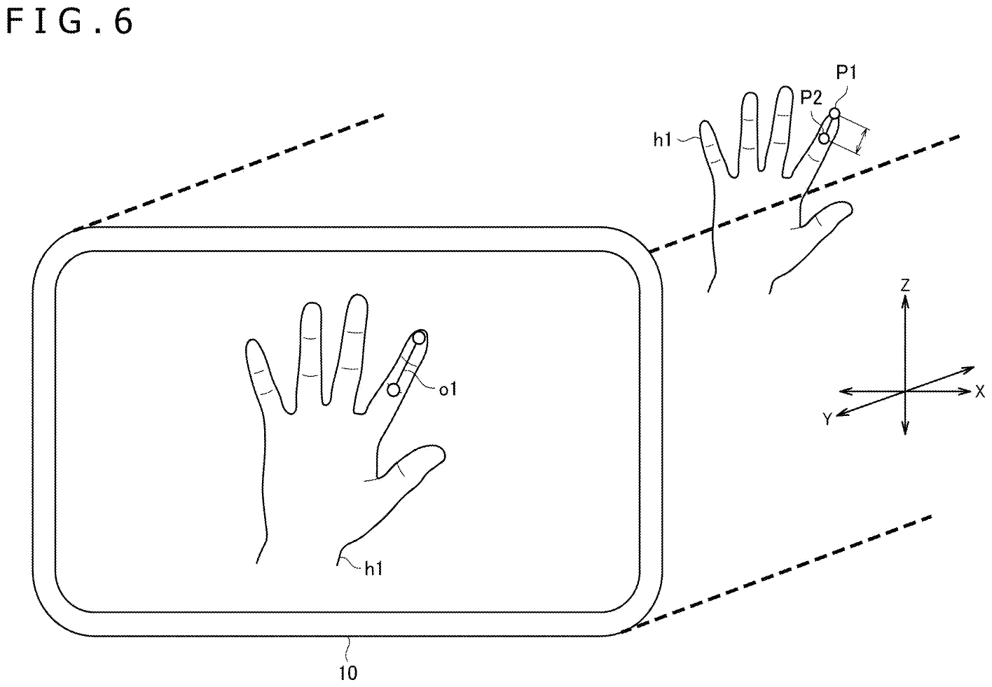

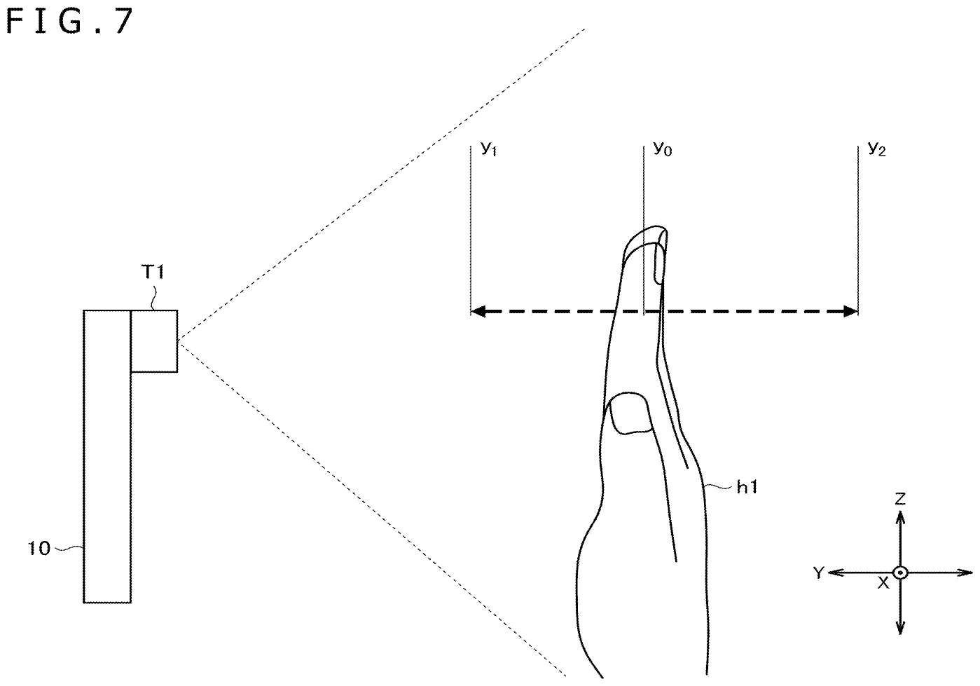

Examples of a method adopted by the model customization processing section 121 to modify the length of a finger of the hand model will now be specifically described with reference to FIGS. 6 and 7.

FIGS. 6 and 7 are explanatory diagrams illustrating the examples of the method adopted by the model customization processing section 121 to modify the length of a target part of the hand model. In FIGS. 6 and 7, an x-axis represents an in-plane longitudinal direction of the information processing device 10, a y-axis represents an out-of-plane direction of the information processing device 10, and a z-axis represents an in-plane transverse direction of the information processing device 10.

In FIG. 6, the information processing device 10 is in a forward direction of the y-axis, whereas the hand h1 of the user is a rear direction of the y-axis. Further, the hand h1 of the user is actually displayed on the display of the information processing device 10.

In the description given with reference to FIG. 6, it is assumed that the desired part of the user to be subjected to length modification is a part (hereinafter referred to as the first part) between the tip of the index finger and a first joint of the index finger. However, the desired part may alternatively be, for example, a part between the first joint and a second joint or a part between the second joint and a third joint. Still alternatively, the desired part may be a finger other than the index finger, the whole hand, or a part other than the hand (e.g., a foot).

As depicted in FIG. 6, the model customization processing section 121 generates a display screen that contains a video image o1 of a target part corresponding to the first part of the user with the length of the target part fixed in the video image. Then, the display of the information processing device 10 displays the display screen.

For example, in a case where the length of the target part depicted in the video image is greater than the actual length of the first part of the user, the user moves the hand h1 of the user forward along the y-axis until the length of the target part in the video image apparently matches the first part of the user.

Subsequently, the model customization processing section 121 modifies the length of the target part of the hand model in reference to the distance between the distance sensor and the first part at a point of time (hereinafter referred to as the first time point) when the length of the target part in the video image apparently matches the first part of the user.

It should be noted that the model customization processing section 121 may recognize, in reference to a user's determination and operation, that the target part in the video image and the first part of the user are matched in length. For example, the user may perform an operation on the display or utter a voice to indicate a determination that the target part in the video image and the first part of the user are apparently matched in length. Further, the model customization processing section 121 may recognize, as the first time point, the point of time when such an apparent length match is found by the user.

Now, with reference to FIG. 7, the following describes a specific example of the method that is adopted by the model customization processing section 121 to modify the length of a target part of the hand model.

First, the model customization processing section 121 generates, for example, a display screen that displays a video image depicting the target part with its length fixed. In this instance, it is assumed that the distance between a distance sensor T1 and the first part is y₀ cm at a point of time (hereinafter referred to as the second time point) when the length of the target part is fixed in the video image.

Here, in a case where the fixed length of the target part depicted in the video image is greater than the actual length of the first part of the user, the user moves the hand h1 of the user leftward along the y-axis until the length of the target part in the video image apparently matches the first part of the user.

Next, the model customization processing section 121 detects a distance of y₁ cm between the distance sensor T1 and the first part at the first time point.

Subsequently, the model customization processing section 121 may modify the length of the target part of the hand model, for example, by multiplying the length of the target part of the hand model by the distance ratio (y₁/y₀) between the first time point and the second time point.

Similarly, in a case where the fixed length of the target part depicted in the video image is smaller than the actual length of the first part of the user, the user moves the hand h1 of the user rightward along the y-axis until the length of the target part in the video image apparently matches the first part of the user.

Next, the model customization processing section 121 detects a distance of y₂ cm between the distance sensor T1 and the first part at the first time point when the length of the target part in the video image matches the length of the first part of the user.

Subsequently, the model customization processing section 121 may modify the length of the target part of the model, for example, by multiplying the length of the target part of the hand model by the distance ratio (y₂/y₀) between the first time point and the second time point.

It should be noted that the model customization processing section 121 may modify the length of the target part of the model by using lens parameters, based on the distance y₁ or the distance y₂ at the first time point and on the fixed length of the target part in the video image.

### (Width Modification of Fingers of Hand Model)

An example of a method adopted by the model customization processing section 121 to modify the widths for fingers of the hand model will now be described.

The model customization processing section 121, which functions as a width estimation section, estimates the widths for the fingers of the hand model in reference to the virtual lines of the fingers that are generated by the image recognition processing section 109.

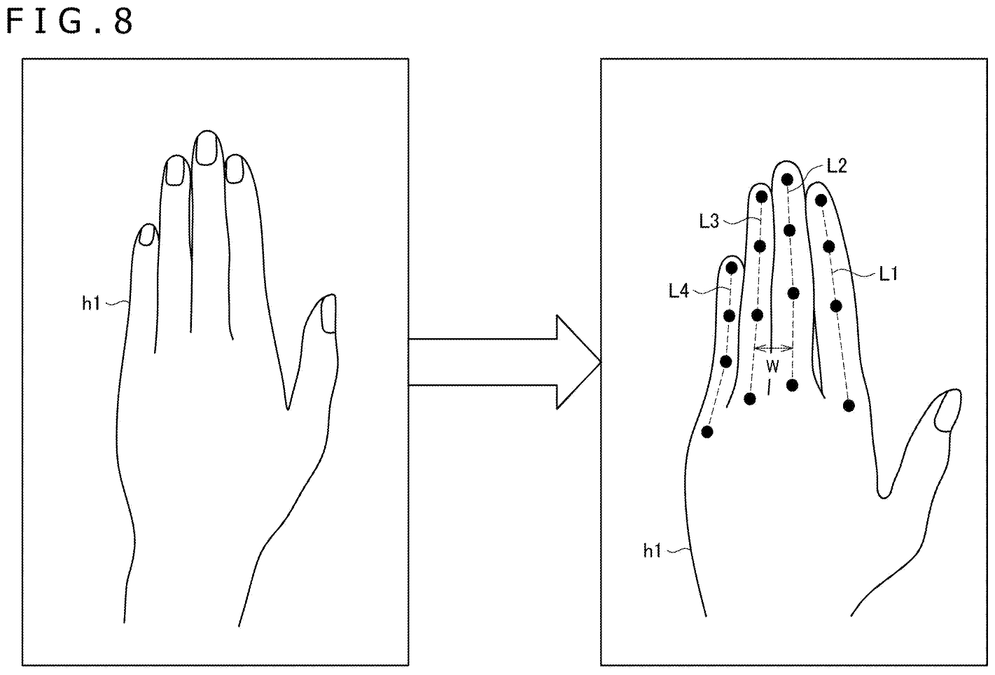

Now, with reference to FIG. 8, the following specifically describes an example of the method adopted by the model customization processing section 121 to modify the widths for the fingers of the hand model.

FIG. 8 is an explanatory diagram illustrating an example of the method adopted by the model customization processing section 121 to modify the widths for the fingers of the hand model. In FIG. 8, the hand h1 of the user is in a state where the fingers other than the thumb are closed.

First, the image recognition processing section 109 detects the joint points of the fingers, and generates virtual lines L1 to L4. Next, the model customization processing section 121 calculates the intervals W between the virtual lines of the fingers sequentially in the longitudinal direction in a state where, for example, a finger is in close contact with adjacent fingers. Then, the model customization processing section 121 estimates the sequentially calculated intervals W as the widths at individual positions in the longitudinal direction of the fingers.

In the case of estimating the widths for the little finger, the model customization processing section 121 may estimate, as the widths for the little finger, the intervals between the virtual line L4 of the little finger and the virtual line L3 of the ring finger adjacent to the little finger.

Further, in the case of estimating the widths for the middle finger, the model customization processing section 121 may estimate, as the widths for the middle finger, the average values of the intervals between the virtual line L2 of the middle finger and the virtual line L1 of the index finger adjacent to the middle finger and the intervals between the virtual line L2 of the middle finger and the virtual line L3 of the ring finger, which is another finger adjacent to the middle finger.

It should be noted that the widths for the index finger can be estimated by a method similar to the method for estimating the widths for the little finger and that the widths for the ring finger can be estimated by a method similar to the method for estimating the widths for the middle finger.

Further, the combination of virtual lines used for estimating the widths for the fingers may be selected as appropriate. For example, in the case of estimating the widths of the ring finger, the model customization processing section 121 need not calculate the widths for the ring finger by determining the average values of the intervals between the ring finger and the middle and little fingers adjacent to the ring finger. For example, the model customization processing section 121 may estimate the widths for the ring finger by determining the intervals between the virtual line L3 of the ring finger and the virtual line L2 of the middle finger.

Further, the example of the method for estimating the widths for the fingers from two-dimensional intervals between the fingers has been described with reference to FIG. 8. Alternatively, however, the model customization processing section 121 may calculate three-dimensional intervals between the fingers, and estimate the widths for the fingers from the calculated three-dimensional intervals between the fingers.

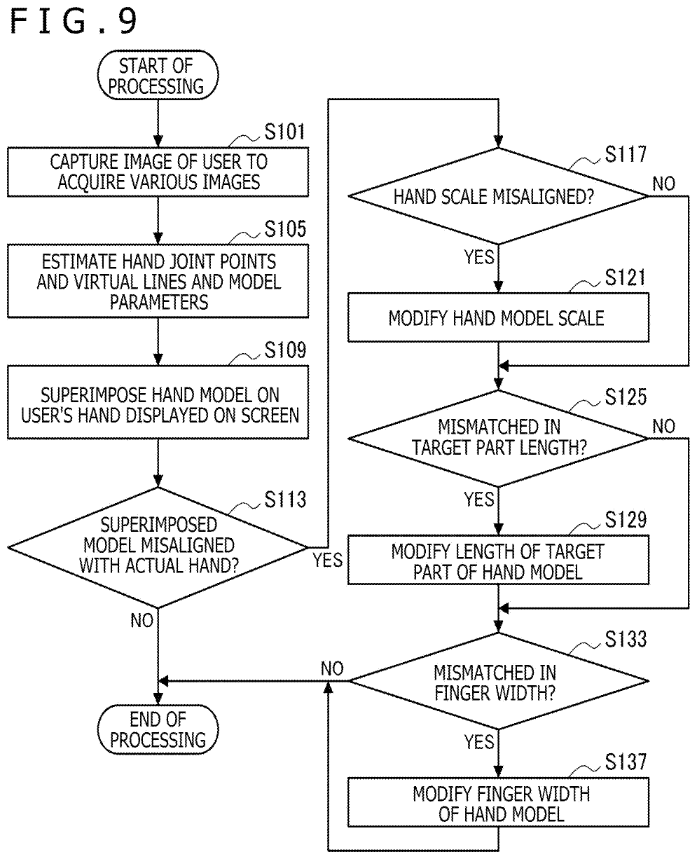

The functional configuration of the information processing device 10 according to the present disclosure has been described thus far. Examples of operation processing performed by the information processing system according to the present disclosure will now be described with reference to FIGS. 9 to 13.

### <3. Examples of Operation Processing>

### (Operations of Information Processing System)

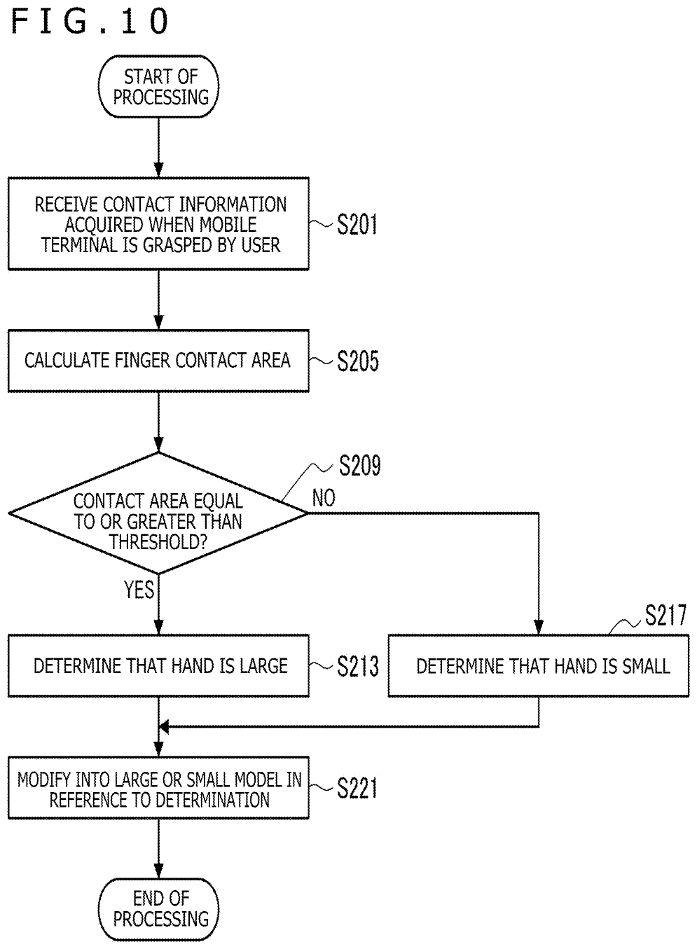

FIG. 9 is an explanatory diagram illustrating the operations of the information processing system according to the present disclosure. Processing performed in each of S121, S129, and S137 will be described in detail later with respective reference to FIGS. 10 to 13.

First, the image/distance information acquisition section 101 acquires RAW data by capturing an image of a hand of the user, and then the image processing section 105 converts the acquired RAW data to various images (S101).

Next, the image recognition processing section 109 detects the joint points of the hand in reference to the various images obtained by conversion performed in S101, and generates virtual lines by connecting the joint points. Then, the model CG creation section 113 estimates the model parameters in reference to the detected joint points of the hand (S103).

Subsequently, in reference to the model parameters estimated in S103, the model CG creation section 113 creates a hand model, and prompts the application section 117 to superimpose the created hand model on the hand of the user, which is displayed on screen (S109).

Next, the application section 117 prompts the user to select whether or not the hand model superimposed in S109 is misaligned with the actual hand (S113). In a case where the user's selection indicates that a misalignment has occurred (S113/Yes), the processing proceeds to S117. In a case where the user's selection indicates that no misalignment has occurred (S113/No), the information processing device 10 terminates the processing.

In the case where the user's selection indicates that a misalignment has occurred (S113/Yes), the model customization processing section 121 prompts the user to select whether or not the scale of the hand model is misaligned with the scale of the hand (S117). In a case where the user's selection indicates that a scale misalignment has occurred (S117/Yes), the processing proceeds to S121. In a case where the user's selection indicates that no scale misalignment has occurred (S117/No), the processing proceeds to S125.

In a case where the user's selection indicates that a hand scale misalignment has occurred (S117/Yes), the model customization processing section 121 modifies the scale of the hand model (S121). Upon completion of S121, the processing proceeds to S125.

In the case where the user's selection indicates that no hand scale misalignment has occurred (S117/No), or after the scale of the hand model is modified (S121), the model customization processing section 121 prompts the user to select whether or not the length of the target part of the hand model matches the length of a part of the user that corresponds to the target part (S125). In a case where the user's selection indicates that the lengths of the target part and the corresponding part of the user mismatch (S125/Yes), the processing proceeds to S129. In a case where the user's selection indicates that the lengths of the target part and the corresponding part of the user do not mismatch (S125/No), the processing proceeds to S133.

In the case where the user's selection indicates that the lengths of the target part and the corresponding part of the user mismatch (S125/Yes), the model customization processing section 121 modifies the length of the target part of the hand model (S129). Upon completion of S129, the processing proceeds to S133.

In the case where the user's selection indicates that the lengths of the target part and the corresponding part of the user do not mismatch (S125/No), or after the length of the target part of the hand model is modified (S121), the model customization processing section 121 prompts the user to select whether or not the finger width of the hand model mismatches the finger width of the hand of the user (S133). In a case where the user's selection indicates that the finger widths mismatch (S133/Yes), the processing proceeds to S137. In a case where the user's selection indicates that the finger widths do not mismatch (S133/No), the information processing device 10 terminates the processing.

In the case where the user's selection indicates that the finger widths mismatch (S133/Yes), the model customization processing section 121 modifies the finger widths of the hand model (S137). Upon completion of step S137, the information processing device 10 terminates the processing.

### (Scale Modification of Hand Model)

FIG. 10 is an explanatory diagram illustrating an example flow for modifying the scale of the hand model by using the first scale modification method according to the present disclosure. First, the information processing device 10 receives the contact information regarding the contact between the touch display and the fingers of the user, which is obtained when the mobile terminal is grasped by the user (S201).

Subsequently, in reference to the received contact information, the model customization processing section 121 calculates the contact area between the touch display and the user's fingers (S205).

Next, the model customization processing section 121 determines whether or not the calculated contact area is equal to or greater than the threshold. In a case where the contact area is equal to or greater than the threshold, the processing proceeds to S213 (S209/Yes). In a case where the contact area is smaller than the threshold, the processing proceeds to S217 (S209/Yes).

In the case where the contact area is equal to or greater than the threshold (S209/Yes), the model customization processing section 121 determines that the user's hand has a large scale (S213).

In the case where the contact area is smaller than the threshold (S209/No), the model customization processing section 121 determines that the user's hand has a small scale (S217).

Subsequently, in reference to the determination made in S213 or S217, the model customization processing section 121 modifies the hand model either into the first scale hand model or into the second scale hand model having a smaller scale than the first scale (S221). More specifically, in a case where it is determined that the user's hand is large (S213), the model customization processing section 121 modifies the hand model into the first scale hand model. Meanwhile, in a case where it is determined that the user's hand is small (S217), the model customization processing section 121 modifies the hand model into the second scale hand model. Subsequently, the information processing device 10 terminates the process of modifying the scale of the hand model.

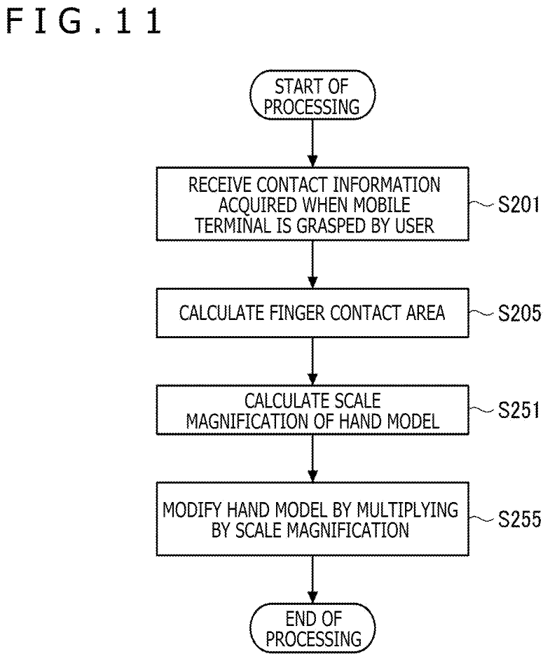

FIG. 11 is an explanatory diagram illustrating an example flow for modifying the scale of the hand model by using the second scale modification method according to the present disclosure. The processing performed in S201 and S205 has been described with reference to FIG. 10. Therefore, S201 and S205 in FIG. 11 will not be redundantly described.

The model customization processing section 121 calculates, as the scale magnification, the ratio between the average contact area of the hand model having a pre-stored desired scale and the contact area calculated in S205 (S251).

Subsequently, the model customization processing section 121 modifies the hand model by multiplying the pre-stored desired scale of the hand model by the scale magnification calculated in S251 (S255). Upon completion of S255, the information processing device 10 terminates the process of modifying the scale of the hand model.

### (Length Modification of Target Part of Hand Model)

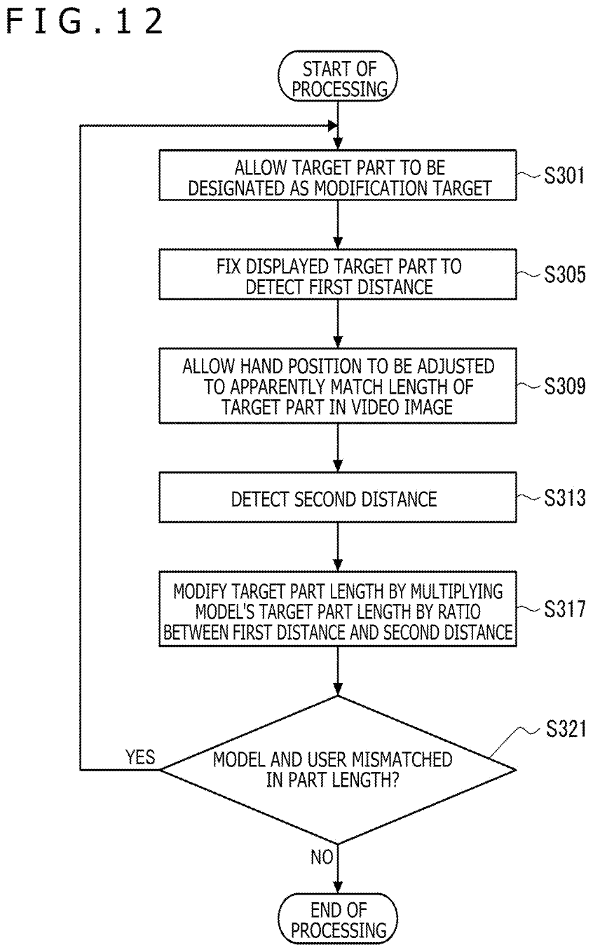

FIG. 12 is an explanatory diagram illustrating an example flow for modifying the length of a target part of the hand model according to the present disclosure. The user performs an operation to designate, as a modification target, a target part of the hand model that corresponds to the first part of a hand of the user (S301).

Next, the model customization processing section 121 generates a display screen in which an image of the target part of the hand model is fixed. Then, the image/distance information acquisition section 101 detects the first distance (S305). The first distance is the distance from the position of the distance sensor to the first part of the user and determined at a point of time when the on-screen image of the target part is fixed.

Subsequently, the user performs an operation for adjusting the position of the user's hand until the length of the target part in the video image apparently matches the length of the first part of the user, and determining that the target part in the video image apparently matches the first part (S309).

Next, the image/distance information acquisition section 101 detects a second distance (S313). The second distance is the distance from the position of the distance sensor to the first part of the user and determined at a point of time when an operation is performed by the user to determine that the length of the target part in the video image apparently matches the length of the first part.

Next, the model customization processing section 121 modifies the length of the target part of the hand model by multiplying the target part of the model by the ratio between the first distance and the second distance (S317).

Next, the model customization processing section 121 prompts the user to select whether or not the lengths of the other parts of the user mismatch the lengths of hand model parts corresponding to the other parts of the user (S321). In a case where the user's selection indicates that the lengths of any corresponding parts mismatch (S321/Yes), the processing returns to S301 to perform a length modification process on a part of the hand model that mismatches a part of the user. In a case where the user's selection indicates that there is no mismatch between the lengths of any parts (S321/No), the information processing device 10 terminates the process of modifying the lengths of the parts of the hand model.

### (Width Modification of Fingers of Hand Model)

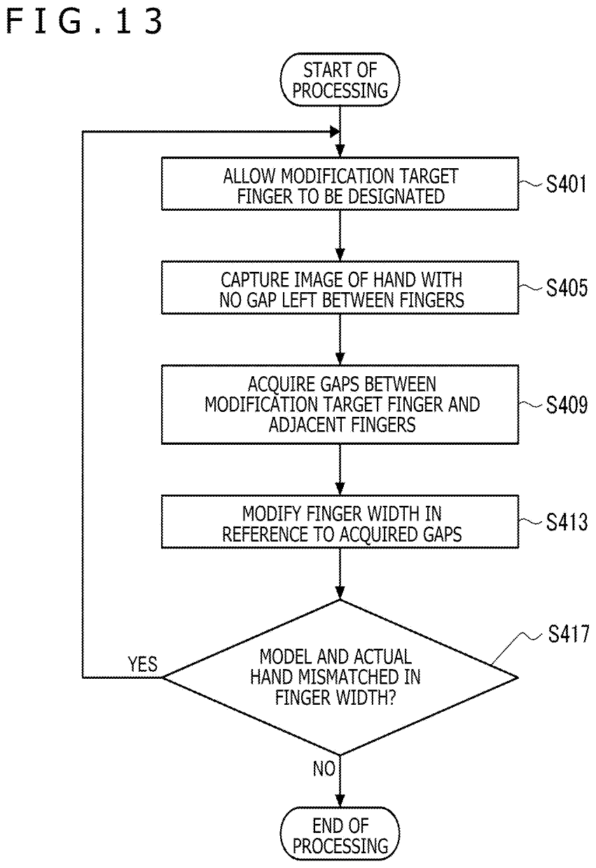

FIG. 13 is an explanatory diagram illustrating an example flow for modifying the width for a finger of the hand model according to the present disclosure. First, the user performs an operation for designating a finger of the hand model that is to be modified (S401).

Next, the image/distance information acquisition section 101 acquires a digital image by capturing an image that depicts a modification target finger and a finger adjacent to the modification target finger while these fingers are in close contact with each other (S405).

Subsequently, the model customization processing section 121 sequentially acquires, from the digital image, the intervals between the virtual line of the modification target finger and the virtual line of the finger adjacent to the modification target finger in the longitudinal direction (S409).

Next, the model customization processing section 121 modifies the width for the modification target finger of the hand model in reference to the acquired intervals (S413).

Next, the model customization processing section 121 prompts the user to select whether or not the widths for the fingers of the hand model mismatch the widths for the corresponding fingers of the user (S417). In a case where there is any mismatch in finger width (S417/Yes), the processing returns to S301 to perform a width modification process on a finger of the hand model that mismatches a corresponding finger of the user. In a case where the user's selection indicates that there is no mismatch in finger width (S417/No), the information processing device 10 terminates the process of modifying the widths for the fingers of the hand model.

The operations of the information processing system according to the present disclosure have been described above. Examples of operational advantages provided by the present disclosure will now be described.

### <4. Examples of Operational Advantages>

The present disclosure, which has been described above, provides various operational advantages. For example, the model customization processing section 121 modifies the length of a target part of the model in reference to the distance between the distance sensor and a part of the user. Hence, this saves the user from having to input parameters and prepare advanced measuring instruments. As a result, the user is able to modify the length of a target part of the model in an easy and simple manner.

Further, the model customization processing section 121 sequentially estimates the width for a specific finger in the longitudinal direction in reference to the intervals between the virtual line of the specific finger and the virtual line of a finger adjacent to the specific finger. This makes it possible to estimate with high accuracy the width of each position that may vary in the longitudinal direction of a finger.

Further, the model customization processing section 121 modifies the scale of the hand model in reference to the contact area that is detected when the mobile terminal is grasped by the user. Hence, the scale of the hand model can be modified, for example, by using a smartphone or other mobile terminal that is owned beforehand by the user.

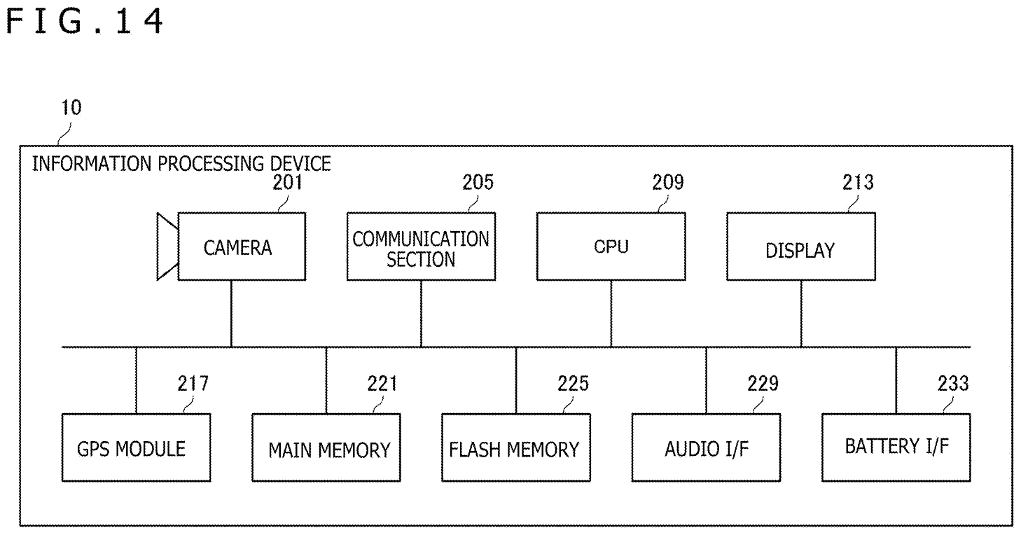

The examples of operational advantages provided by the present disclosure have been described above. An example hardware configuration of the information processing device 10 according to the present disclosure will now be described with reference to FIG. 14.

<5. Example Hardware Configuration of Information Processing Device 10 according to Present Disclosure>

FIG. 14 is a block diagram illustrating an example hardware configuration of the information processing device 10 according to the present disclosure. The information processing device 10 may include a camera 201, a communication section 205, a CPU (Central Processing Unit) 209, a display 213, a GPS (Global Positioning System) module 217, a main memory 221, a flash memory 225, an audio interface 229, and a battery interface 233.

The camera 201 is configured to represent an example of the image/distance information acquisition section 101 according to the present disclosure. The camera 201 captures an image of the subject to acquire an electrical signal containing the image information or the distance information.

The communication section 205 receives data, for example, from an additional mobile device, and transmits a model modified in reference to the received data to the additional mobile device.

The CPU 209, which functions as an arithmetic processing unit and as a control device, controls overall operation in the information processing device 10, according to various programs. Further, the CPU 209 is able to implement the functions, for example, of the image processing section 105, the model CG creation section 113, and the model customization processing section 121 by collaborating with the later-described main memory 211, flash memory 215, and software.

The display 213 is, for example, a display device such as a CRT (Cathode Ray Tube) display device, a liquid-crystal display (LCD), or an OLED (Organic Light Emitting Diode) device, and configured to convert video data to a video image and output the resulting video image. The display 213 may display, for example, a display screen that displays a video image depicting a target part with its length fixed.

The GPS module 217 measures, for example, the latitude, longitude, or altitude of the information processing device 10 by using a GPS signal received from a GPS satellite. For example, using information measured by the GSP signal enables the model customization processing section 121 to calculate the intervals between the fingers from three-dimensional position information regarding the individual fingers that includes latitude, longitude, or altitude.

The main memory 221 temporarily stores, for example, a program to be executed by the CPU 209 and parameters that vary as appropriate from one program execution to another. The flash memory 225 stores, for example, a program and arithmetic parameters to be used by the CPU 209.

The CPU 209, the main memory 221, and the flash memory 225 are interconnected by an internal bus, and further connected through an input/output interface to the communication section 205, the display 213, the GPS module 217, the audio interface 229, and the battery interface 233.

The audio interface 229 is for connecting to speakers, earphones, and other sound generating devices. The battery interface 233 is for connecting to a battery or a battery-equipped device.

### <6. Supplement>

While the preferred embodiment of the present disclosure has been described in detail with reference to the accompanying drawings, the technical scope of the present disclosure is not limited to the above-described preferred embodiment. It is obvious that persons having ordinary knowledge of the technical field of the present disclosure are able to easily conceive of various alterations or modifications within the scope of technical ideas described in the appended claims. Accordingly, it is to be understood that such alterations and modifications are also within the technical scope of the present disclosure.

For example, the model customization processing section 121 may have only the function of modifying one or two of the parameters representing the scale of the hand model, the length of a target part of the model, and the widths for fingers of the hand model.

Further, when adjusting the length of a target part, the model customization processing section 121 may add an offset to a depth image between the position of the distance sensor and a part of the user, and then modify the length of the target part of the model. For example, in a case where the length of the part of the user is greater than the length of the target part in the video image, the model customization processing section 121 may add, to the depth image, an offset oriented in the direction of bringing a hand closer. Subsequently, the model customization processing section 121 may modify the length of the target part of the model in reference to the amount of the added offset. This eliminates the necessity of adjusting the position of the hand until it apparently matches the length of the target part in the video image. Thus, the burden on the user can be reduced.

Further, the processing steps for the operation of the information processing device 10 according to the present disclosure need not necessarily be performed chronologically in the order depicted in the explanatory diagrams. For example, the individual processing steps for the operation of the information processing device 10 may be performed in an order different from the order depicted in the explanatory diagrams or may be parallelly performed.

Moreover, a computer program may be created to enable the hardware built in the information processing device 10, such as the CPU, the ROM, and the RAM, to implement functions equivalent to the functions of the above-described constituent elements included in the information processing device 10.

Additionally, the advantages described in this document are merely descriptive or illustrative and not restrictive. Stated differently, the technology according to the present disclosure is able to provide advantages obvious to persons skilled in the art from the description in this document in addition to or in place of the above-described advantages.

It should be noted that the following configurations are also within the technical scope of the present disclosure.
(1)
   An information processing device including:
   a display control section that generates a display screen containing a video image of a target part, the target part being one of parts included in a model, being fixed in length in the video image, and corresponding to a first part of a user; and
   a modification section that modifies a length of the target part of the model in reference to a first distance at a first time point, the first distance being determined by a distance sensor and indicative of a distance between the distance sensor and the first part of the user, and the first time point being a point of time when the video image of the target part apparently matches a length of the first part of the user.
(2)
   The information processing device according to (1) above, in which the modification section modifies the length of the target part of the model in reference to the first distance and a second distance at a second time point, the second distance being a distance between a position of the distance sensor and a second part, and the second time point being a point of time when the video image of the target part is fixed.
(3)
   The information processing device according to (2) above, in which the modification section modifies the length of the target part of the model in reference to a ratio between the first distance and the second distance.
(4)
   The information processing device according to any one of (1) through (3) above, in which the model is a hand model, and
   the first part is one of a part between a tip of a finger and a first joint of the finger, a part between the first joint and a second joint of the finger, and a part between the second joint and a third joint of the finger.
(5)
   The information processing device according to any one of (1) through (4) above, further including:
   a detection section that detects joint points from each of multiple fingers of the user;
   a virtual line generation section that generates a virtual line for each of the multiple fingers, the virtual line sequentially connecting the detected joint points; and
   a width estimation section that estimates a width for a first finger of the multiple fingers in reference to an interval between the virtual line corresponding to the first finger and the virtual line corresponding to a second finger adjacent to the first finger in a state where the first finger is in close contact with the second finger.
(6)
   The information processing device according to (5) above, in which the width estimation section estimates the width for the first finger of the multiple fingers in reference to the interval between the virtual line corresponding to the first finger and the virtual line corresponding to the second finger adjacent to the first finger and an interval between the virtual line corresponding to the first finger and the virtual line corresponding to a third finger adjacent to the first finger, in a state where the first finger is in close contact with the second finger and the third finger.
(7)
   The information processing device according to any one of (4) through (6) above, further including:
   a contact area calculation section that calculates a contact area of a specific part of the user that is detected when a mobile terminal is grasped by the user,
   in which the display control section generates a display screen that contains a video image of a hand model having a size corresponding to the contact area.
(8)
   The information processing device according to (7) above, in which the display control section generates a display screen containing a video image of a hand model having a first scale when the contact area is equal to or greater than a threshold, and generates a display screen containing the video image of the hand model having a second scale when the contact area is smaller than the threshold, the second scale being smaller than the first scale.
(9)
   The information processing device according to (7) above, further including:
   a storage section that pre-stores a desired hand model and an average contact area of the specific part with respect to the desired hand model; and
   a magnification calculation section that calculates a scale magnification in reference to the contact area calculated by the contact area calculation section and the average contact area stored by the storage section,
   in which the display control section generates a display screen containing an image of a hand model that is drawn by multiplying a scale value of the desired hand model by the scale magnification.
(10)
   The information processing device according to any one of (7) through (9) above, in which the specific part is a finger pad of the user.
(11)
   An information processing method executed by a computer, including:
   generating a display screen that contains a video image of a target part, the target part being one of parts included in a model, being fixed in length in the video image, and corresponding to a first part of a user; and
   modifying a length of the target part of the model in reference to a first distance at a first time point, the first distance being determined by a distance sensor and indicative of a distance between the distance sensor and the first part of the user, and the first time point being a point of time when the video image of the target part apparently matches a length of the first part of the user.
(12)
   An information processing program that causes a computer to function as:
   a display control section that generates a display screen containing a video image of a target part, the target part being one of parts included in a model, being fixed in length in the video image, and corresponding to a first part of a user; and
   a modification section that modifies a length of the target part of the model in reference to a first distance at a first time point, the first distance being determined by a distance sensor and indicative of a distance between the distance sensor and the first part of the user, and the first time point being a point of time when the video image of the target part apparently matches a length of the first part of the user.

### REFERENCE SIGNS LIST

10 Information processing device
101: Image/distance information acquisition section
105: Image processing section
109: Image recognition processing section
113: Model CG creation section
117: Application section
121: Model customization processing section

1. An information processing device comprising:
a display control section that generates a display screen containing a video image of a target part, the target part being one of parts included in a model, being fixed in length in the video image, and corresponding to a first part of a user; and
a modification section that modifies a length of the target part of the model in reference to a first distance at a first time point, the first distance being determined by a distance sensor and indicative of a distance between the distance sensor and the first part of the user, and the first time point being a point of time when the video image of the target part apparently matches a length of the first part of the user.

2. The information processing device according to claim 1, wherein the modification section modifies the length of the target part of the model in reference to the first distance and a second distance at a second time point, the second distance being a distance between a position of the distance sensor and a second part, and the second time point being a point of time when the video image of the target part is fixed.

3. The information processing device according to claim 2, wherein the modification section modifies the length of the target part of the model in reference to a ratio between the first distance and the second distance.

4. The information processing device according to claim 3, wherein the model is a hand model, and
the first part is one of a part between a tip of a finger and a first joint of the finger, a part between the first joint and a second joint of the finger, and a part between the second joint and a third joint of the finger.

5. The information processing device according to claim 4, further comprising:
a detection section that detects joint points from each of multiple fingers of the user;
a virtual line generation section that generates a virtual line for each of the multiple fingers, the virtual line sequentially connecting the detected joint points; and
a width estimation section that estimates a width for a first finger of the multiple fingers in reference to an interval between the virtual line corresponding to the first finger and the virtual line corresponding to a second finger adjacent to the first finger in a state where the first finger is in close contact with the second finger.

6. The information processing device according to claim 5, wherein the width estimation section estimates the width for the first finger of the multiple fingers in reference to the interval between the virtual line corresponding to the first finger and the virtual line corresponding to the second finger adjacent to the first finger and an interval between the virtual line corresponding to the first finger and the virtual line corresponding to a third finger adjacent to the first finger, in a state where the first finger is in close contact with the second finger and the third finger.

7. The information processing device according to claim 6, further comprising:
a contact area calculation section that calculates a contact area of a specific part of the user that is detected when a mobile terminal is grasped by the user,
wherein the display control section generates a display screen that contains a video image of a hand model having a size corresponding to the contact area.

8. The information processing device according to claim 7, wherein the display control section generates a display screen containing a video image of a hand model having a first scale when the contact area is equal to or greater than a threshold, and generates a display screen containing the video image of the hand model having a second scale when the contact area is smaller than the threshold, the second scale being smaller than the first scale.

9. The information processing device according to claim 7, further comprising:
a storage section that pre-stores a desired hand model and an average contact area of the specific part with respect to the desired hand model; and
a magnification calculation section that calculates a scale magnification in reference to the contact area calculated by the contact area calculation section and the average contact area stored by the storage section,
wherein the display control section generates a display screen containing an image of a hand model that is drawn by multiplying a scale value of the desired hand model by the scale magnification.

10. The information processing device according to claim 9, wherein the specific part is a finger pad of the user.

11. An information processing method executed by a computer, comprising:
generating a display screen that contains a video image of a target part, the target part being one of parts included in a model, being fixed in length in the video image, and corresponding to a first part of a user; and
modifying a length of the target part of the model in reference to a first distance at a first time point, the first distance being determined by a distance sensor and indicative of a distance between the distance sensor and the first part of the user, and the first time point being a point of time when the video image of the target part apparently matches a length of the first part of the user.

12. An information processing program that causes a computer to function as:
a display control section that generates a display screen containing a video image of a target part, the target part being one of parts included in a model, being fixed in length in the video image, and corresponding to a first part of a user; and
a modification section that modifies a length of the target part of the model in reference to a first distance at a first time point, the first distance being determined by a distance sensor and indicative of a distance between the distance sensor and the first part of the user, and the first time point being a point of time when the video image of the target part apparently matches a length of the first part of the user.
